(19) United States
(12) Patent Application Publication
(10) Pub. No.: US 2007/0011825 A1
(43) Pub. Date: Jan. 18, 2007

(54) Novel double para-phenylenediamines joined by a linkage comprising a saturated cyclic radical and use in dyeing

(76) Inventors: Stephane Sabelle, Paris (FR); Eric Metais, St Leu la Foret (FR); Xavier Radisson, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/476,816

(22) Filed: Jun. 29, 2006

Related U.S. Application Data
(60) Provisional application No. 60/698,935, filed on Jul. 14, 2005.

(30) Foreign Application Priority Data
Jun. 29, 2005 (FR) 05 51805

(51) Int. Cl. A61K 8/00

(52) U.S. Cl. 8/405

(57) ABSTRACT

The present application relates to a novel family of double para-phenylenediamines joined by a linkage comprising a saturated cyclic radical and use thereof for the dyeing of keratin fibers. These novel para-phenylenediamines can be used as an oxidation base for the dyeing of keratin fibers. Methods of making and using, and a dyeing kit are also disclosed.

This application claims benefit of U.S. Provisional Application No. 60/698,935, filed Jul. 14, 2005, the contents of which are incorporated herein by reference. This application also claims benefit of priority under 35 U.S.C. § 119 to French Patent Application No. FR 05 51805, filed Jun. 29, 2005, the contents of which are also incorporated herein by reference.

The present application relates to a novel family of double para-phenylenediamines joined by a linkage comprising a saturated cyclic radical and use thereof for the dyeing of keratin fibers.

The dyeing of keratin fibers, such as human hair, with dyeing compositions comprising oxidation dye precursors, generally called oxidation bases, such as ortho- or para-phenylenediamines, ortho- or para-aminophenols and heterocyclic compounds, is known in the art. These oxidation bases are colorless or weakly colored compounds, which when combined with oxidizing products can give rise, by a process of oxidative condensation, to colored compounds.

It is also known that it is possible to vary the shades obtained with these oxidation bases by combining them with couplers or dyeing modifiers, the latter being chosen, for example, from aromatic meta-diaminobenzenes, meta-aminophenols, meta-diphenols and some heterocyclic compounds, such as indole compounds and pyridine compounds.

The great variety of the molecules employed for the oxidation bases and the couplers means that a rich palette of colors can be obtained.

In addition, the so-called “permanent” dyeing obtained using these oxidation dyes should ideally satisfy a number of requirements. Thus, the permanent dye ideally presents no problems of a toxicological nature, makes it possible to obtain the color shades of the desired intensity, has good resistance to external agents such as light, weather, washing, permanent waving, sweat and/or rubbing, provides coverage of white hair, and/or displays minimum selectivity, i.e., ensures that the smallest possible differences in coloration are obtained all the way along the same keratin fiber, which generally is differently sensitized (i.e. damaged) between its tip and its root.

The present application discloses novel oxidation bases capable of dyeing keratin fibers in a variety of strong, aesthetic shades with low selectivity, as well as colors that are resistant to the various aggressive factors to which the fibers may be subjected, such as light, sweat and shampoos.

The present disclosure thus relates to a novel family of double para-phenylenediamines of the following formula (I) as well as the corresponding addition salts:

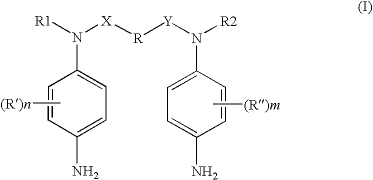

in which:
R is a saturated ring with 4 to 7 ring members, optionally comprising at least one atom chosen from nitrogen and oxygen atoms; wherein said ring is optionally substituted, for example, with at least one C₁-C₄ alkyl group,
R₁ and R₂ are, independently of one another, chosen from:

hydrogen,

linear or branched C₁-C₆ alkyl radicals,

linear or branched C₁-C₆ alkyl radicals substituted with at least one radical chosen from hydroxy, C₁-C₄ alkoxy, amino, C₁-C₄ monoalkylamino, and C₁-C₄ dialkylamino radicals,
X and Y are, independently of one another, chosen from linear or branched C₁-C₁₀ alkylene radicals and a covalent bond,
R′ and R″ are, independently of one another, chosen from:

C₁-C₆ alkyl radicals,

C₁-C₆ alkoxy radicals,

C₁-C₆ hydroxy-alkoxy radicals,

C₁-C₆ alkoxy(C₁-C₆)alkyl radicals,

C₁-C₆ mono- or poly-hydroxy alkyl radicals,
n and m are, independently of one another, integers from 0 to 4 with the exception of the following compound:

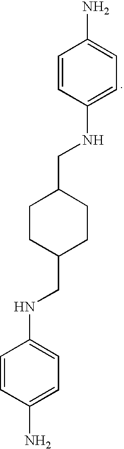

The present disclosure also relates to compositions for the dyeing of keratin fibers, for instance human keratin fibers, such as the hair, comprising at least one para-phenylenediamine of formula (I). The composition of the present disclosure makes it possible for instance to obtain very strong coloration of keratin fibers, of low selectivity and/or resistant, for example, to light.

The present disclosure also relates to a method of dyeing keratin fibers, for instance human keratin fibers such as the hair, by applying to the keratin fibers a composition as disclosed herein and to a device with several compartments or dyeing “kit”.

The following para-phenylenediamines may be mentioned as non-limiting examples:

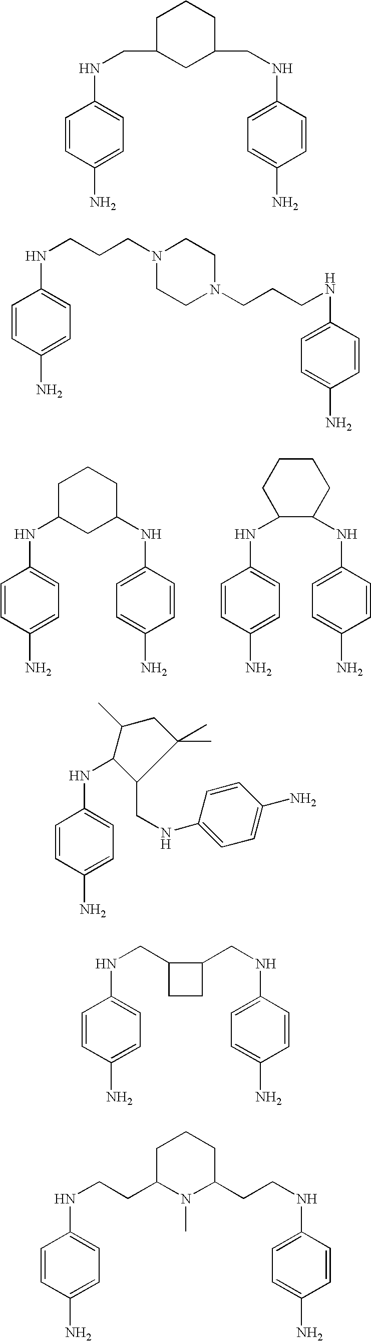

According to at least one embodiment, the para-phenylenediamines of formula (I) are such that R is a saturated cyclic radical optionally comprising at least one nitrogen atom. As a substituent on R, non-limiting mention may be made of C₁-C₄ alkyl groups. In at least one embodiment, R₁ and R₂ are independently chosen from: hydrogen and C₁-C₄ alkyl groups that can be substituted. In at least one embodiment, X and Y are independently chosen from: C₁-C₃ alkylene radicals and a covalent bond. And further, in at least one embodiment, n and m are equal to 0 or 1.

In general, the addition salts that can be used herein may, for example, be chosen from acid-addition salts of, for example, hydrochloric acid, hydrobromic acid, sulphuric acid, citric acid, succinic acid, tartaric acid, lactic acid, para-toluene-sulphonic acid, benzene-sulphonic acid, phosphoric acid and acetic acid.

They can also be in the form of solvates, for example a hydrate or a solvate of a linear or branched alcohol such as ethanol or isopropanol.

The para-phenylenediamines of formula (I) according to the present disclosure can be prepared according to a conventional method of synthesis. Reference may be made for example to Patent Application DE-10144226 A.

By way of illustration, the para-phenylenediamines of formula (I) can be synthesized according to the following reaction scheme:

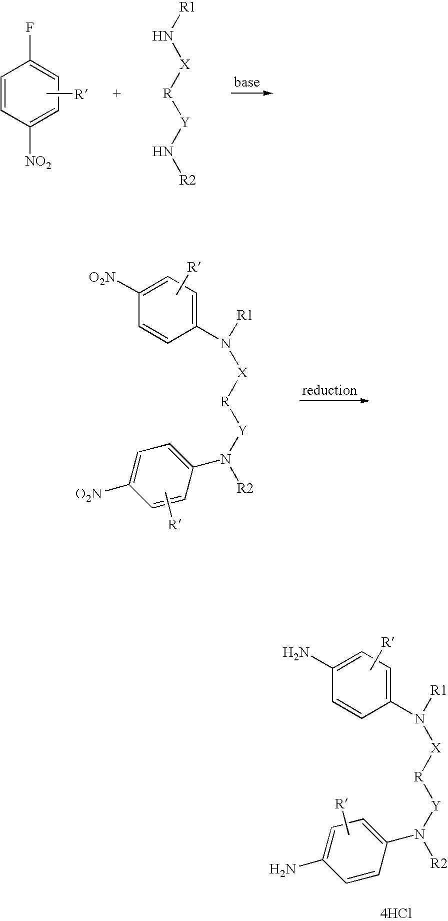

The first stage in the synthesis is a nucleophilic substitution of a diamine on a derivative of para-fluoro-nitrobenzene, a stage suggested by the publications Synthesis 1990 (12), 1147-1148 and Synth. Commun. 1990, 20 (22), 3537-3545. The second stage is a conventional reduction stage, and can be, for example, a reaction of hydrogenation by heterogeneous catalysis in the presence of Pd/C, Pd(II)/C, Raney Nickel or a reaction of reduction by a metal, for example by zinc, iron, tin etc. (Advanced Organic Chemistry, 4th edition, 1992, J. March, Wiley Interscience; Reduction in Organic Chemistry, M. Hudlicky, 1983, Ellis Horwood Series Chemical Science).

Also disclosed herein are nitrogen-containing compounds of the following formula (II) which can be used for obtaining para-phenylenediamines of formula (I):

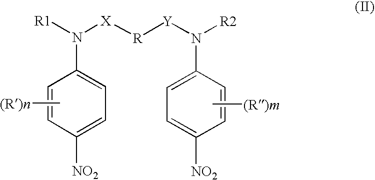

in which R₁, R₂, X, Y, R, R′, R″, n and m are as defined above for formula (I).

The present disclosure also relates to a dyeing composition comprising, in a medium that is suitable for dyeing, at least one oxidation base chosen from para-phenylenediamine compounds of formula (I) as defined above, and addition salts thereof, and this composition may include the compound:

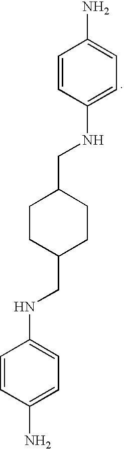

The amount of para-phenylenediamine of formula (I) in the dyeing composition ranges from 0.0001 wt. % to 20 wt. % relative to the total weight of the composition, for example from 0.01% to 10 wt. % relative to the total weight of the composition.

According to at least one embodiment, the composition as disclosed herein comprises at least one oxidation coupler.

Among the oxidation couplers useful herein, non-limiting mention may be made of meta-phenylenediamines, meta-aminophenols, meta-diphenols, naphthalenic couplers. heterocyclic couplers, and addition salts thereof.

By way of non-limiting examples, mention may be made of: 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diamino-benzene, 1,3-bis-(2,4-diaminophenoxy)propane, 3-ureido-aniline, 3-ureido-1-dimethylaminobenzene, sesamol, 1-β-hydroxyethylamino-3,4-methylene-dioxybenzene, α-naphthol, 2-methyl-1-naphthol, 6-hydroxy-indole, 4-hydroxy-indole, 4-hydroxy-N-methyl indole, 2-amino-3-hydroxypyridine, 6-hydroxybenzomorpholine, 3,5-diamino-2,6-dimethoxypyridine, 1-N-(β-hydroxyethyl)amino-3,4-methylene dioxybenzene, 2,6-bis-(β-hydroxyethylamino)toluene and their addition salts.

The at least one oxidation coupler is present in an amount ranging from 0.0001 to 20 wt. % relative to the total weight of the composition, for instance from 0.005 to 6 wt. % relative to the total weight of the composition.

The composition according to the present disclosure can also comprise at least one additional oxidation base different from the oxidation bases of formula (I).

The at least one additional oxidation bases can, for example, be chosen from para-phenylenediamines, bis-phenylalkylenediamines, para-aminophenols, ortho-aminophenols, heterocyclic bases, and addition salts thereof.

Among the para-phenylenediamines, non-limiting mention includes: para-phenylenediamine, para-toluenediamine, 2-chloro-para-phenylenediamine, 2,3-dimethyl para-phenylenediamine, 2,6-dimethyl para-phenylenediamine, 2,6-diethyl para-phenylenediamine, 2,5-dimethyl para-phenylenediamine, N,N-dimethyl para-phenylenediamine, N,N-diethyl para-phenylenediamine, N,N-dipropyl para-phenylenediamine, 4-amino-N,N-diethyl-3-methyl aniline, N,N-bis-(β-hydroxyethyl)para-phenylenediamine, 4-N,N-bis-(β-hydroxyethyl)amino-2-methyl aniline, 4-N,N-bis(β-hydroxyethyl)amino-2-chloro-aniline, 2-β-hydroxyethyl para-phenylenediamine, 2-fluoro para-phenylenediamine, 2-isopropyl para-phenylenediamine, N-(β-hydroxypropyl)para-phenylenediamine, 2-hydroxymethyl para-phenylenediamine, N,N-dimethyl-3-methyl para-phenylenediamine, N,N-(ethyl, β-hydroxyethyl)para-phenylenediamine, N-(β,γ-dihydroxypropyl)para-phenylenediamine, N-(4′-aminophenyl)para-phenylenediamine, N-phenyl para-phenylenediamine, 2-β-hydroxyethyloxy para-phenylenediamine, 2-β-acetylaminoethyloxy para-phenylenediamine, N-(β-methoxyethyl)para-phenylenediamine, 2-thienyl para-phenylenediamine, 2-β-hydroxyethylamino-5-aminotoluene, 3-hydroxy-1-(4′-aminophenyl)pyrrolidone, 6-(4-amino-phenylamino)-hexan-1-ol and their acid-addition salts.

Among the para-phenylenediamines mentioned above, para-phenylenediamine, para-toluenediamine, 2-isopropyl para-phenylenediamine, 2-β-hydroxyethyl para-phenylenediamine, 2-β-hydroxyethyloxy para-phenylenediamine, 2,6-dimethyl para-phenylenediamine, 2,6-diethyl para-phenylenediamine, 2,3-dimethyl para-phenylenediamine, N,N-bis-(β-hydroxyethyl)para-phenylenediamine, 2-chloro para-phenylenediamine, 2-α-acetylaminoethyloxy para-phenylenediamine, 6-(4-amino-phenylamino)-hexan-1-ol and their acid-addition salts, may be used, for example.

Among the bis-phenylalkylenediamines, non-limiting examples include: N,N′-bis-(β-hydroxyethyl) N,N′-bis-(4′-aminophenyl) 1,3-diamino-propanol, N,N′-bis(β-hydroxyethyl) N,N′-bis(4′-aminophenyl)ethylenediamine, N,N′-bis-(4′-aminophenyl)tetramethylenediamine, N,N′-bis-(β-hydroxyethyl) N,N′-bis-(4′-aminophenyl)tetramethylenediamine, N,N′-bis-(4′-amino-3′-methylphenyl)tetramethylenediamine, N,N′-bis-(ethyl) N,N′-bis-(4′-amino, 3′-methylphenyl)ethylenediamine, 1,8-bis-(2,5-diamino-phenoxy)-3,6-dioxaoctane, and their acid-addition salts.

Among the para-aminophenols, non-limiting mention may be made of: para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl aminomethyl)phenol, 4-amino-2-fluorophenol, 4-amino-2,6-dichlorophenol, 4-amino-6[((5′-amino-2′-hydroxy-3′-methyl)phenyl)methyl]-2-methylphenol, bis(5′-amino-2′-hydroxy)phenylmethane and their acid-addition salts.

Among the ortho-aminophenols, non-limiting mention may be made of: 2-aminophenol, 2-amino-5-methylphenol, 2-amino-6-methylphenol, 5-acetamido-2-aminophenol, 5-[(2-hydroxyethyl)amino]2-methylphenol and their acid-addition salts.

Among the heterocyclic bases, non-limiting mention may be made of: pyridine derivatives, pyrimidine derivatives and pyrazole derivatives.

Among the pyridine derivatives, non-limiting mention may be made of the compounds described for example in British Patents GB 1 026 978 and GB 1 153 196, as well as 2,5-diamino pyridine, 2-(4-methoxyphenyl)amino-3-amino pyridine, 3,4-diamino pyridine, and their acid-addition salts.

Other pyridine oxidation bases that can be used in the present disclosure are the oxidation bases 3-amino pyrazolo-[1,5-a]-pyridines or their salts of addition described for example in French Patent Application FR 2 801 308. As examples, non-limiting mention may be made of: pyrazolo[1,5-a]pyridin-3-ylamine; 2-acetylamino pyrazolo-[1,5-a]pyridin-3-ylamine; 2-morpholin-4-yl-pyrazolo[1,5-a]pyridin-3-ylamine; 3-amino-pyrazolo[1,5-a]pyridine-2-carboxylic acid; 2-methoxy-pyrazolo[1,5-a]pyridin-3-ylamine; (3-amino-pyrazolo[1,5-a]pyridin-7-yl)-methanol; 2-(3-amino-pyrazolo[1,5-a]pyridin-5-yl)-ethanol; 2-(3-amino-pyrazolo[1,5-a]pyridin-7-yl)-ethanol; (3-amino-pyrazolo[1,5-a]pyridin-2-yl)-methanol; 3,6-diamino-pyrazolo[1,5-a]pyridine; 3,4-diamino-pyrazolo[1,5-a]pyridine; pyrazolo[1,5-a]pyridine-3,7-diamine; 7-morpholin-4-yl-pyrazolo[1,5-a]pyridin-3-ylamine; pyrazolo[1,5-a]pyridine-3,5-diamine; 5-morpholin-4-yl-pyrazolo[1,5-a]pyridin-3-ylamine; 2-[(3-amino-pyrazolo[1,5-a]pyridin-5-yl)-(2-hydroxyethyl)-amino]-ethanol; 2-[(3-amino-pyrazolo[1,5-a]pyridin-7-yl)-(2-hydroxyethyl)-amino]-ethanol; 3-amino-pyrazolo[1,5-a]pyridin-5-ol; 3-amino-pyrazolo[1,5-a]pyridin-4-ol; 3-amino-pyrazolo[1,5-a]pyridin-6-ol; 3-amino-pyrazolo[1,5-a]pyridin-7-ol; as well as their acid-addition salts.

Among the pyrimidine derivatives, non-limiting mention may be made of the compounds described for example in Patents DE 23 59 399; JP 88-169571; JP 05-63124; EP 0 770 375 or Patent Application WO 96/15765 such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their acid-addition salts and their tautomeric forms, when a tautomeric equilibrium exists.

Among the pyrazole derivatives, non-limiting mention may be made of the compounds described in Patents DE 38 43 892, DE 41 33 957 and patent applications WO 94/08969, WO 94/08970, FR-A-2 733 749 and DE 195 43 988 such as 4,5-diamino-1-methyl pyrazole, 4,5-diamino-1-(β-hydroxyethyl)pyrazole, 3,4-diamino pyrazole, 4,5-diamino-1-(4′-chlorobenzyl)pyrazole, 4,5-diamino-1,3-dimethylpyrazole, 4,5-diamino-3-methyl-1-phenyl pyrazole, 4,5-diamino-1-methyl-3-phenyl pyrazole, 4-amino-1,3-dimethyl-5-hydrazino pyrazole, 1-benzyl-4,5-diamino-3-methylpyrazole, 4,5-diamino-3-tert-butyl 1-methyl pyrazole, 4,5-diamino-1-tert-butyl 3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropyl pyrazole, 4,5-diamino-3-methyl-1-isopropyl pyrazole, 4-amino-5-(2′-aminoethyl)amino-1,3-dimethyl pyrazole, 3,4,5-triamino pyrazole, 1-methyl-3,4,5-triamino pyrazole, 3,5-diamino-1-methyl-4-methylamino pyrazole, 3,5-diamino-4-(β-hydroxyethyl)amino-1-methylpyrazole, and their addition salts.

The at least one additional oxidation base is present in the composition in an amount ranging from 0.0001 to 20 wt. %, for instance, ranging from 0.005 to 6 wt. % relative to the total weight of the composition.

The dyeing composition according to the present disclosure can additionally contain at least one direct dye which can be chosen, for example, from neutral, acid or cationic nitrogen-containing dyes of the benzene series, neutral, acid or cationic direct azo dyes, neutral, acid or cationic quinone and for instance, anthraquinone direct dyes, azine direct dyes, methine, azomethine, triarylmethane and indoamine direct dyes and natural direct dyes. In at least one embodiment, the composition according to the present disclosure comprises at least one dye chosen from cationic direct dyes and natural direct dyes.

Among the cationic direct dyes that can be used according to the present disclosure, non-limiting mention may be made of the cationic azo direct dyes described in Patent Applications WO 95/15144, WO-95/01772 and EP-714 954.

Among these compounds, the following dyes may be mentioned as non-limiting examples:
1,3-dimethyl-2-[[4-(dimethylamino)phenyl]azo]-1H-imidazolium chloride,
1,3-dimethyl-2-[(4-aminophenyl)azo]-1H-imidazolium chloride,
1-methyl-4-[(methylphenylhydrazono)methyl]-pyridinium methylsulphate.

Among the natural direct dyes that can be used according to the present disclosure, non-limiting mention may be made of: lawsone, juglone, alizarin, purpurin, carminic acid, kermesic acid, purpurogallin, protocatechaldehyde, indigo, isatin, curcumine, spinulosine, apigenidine. It is also possible to use extracts or decoctions comprising these natural dyes and, in at least one embodiment, henna-based cataplasms or extracts.

The direct dye or dyes may be present in the compositions disclosed herein in an amount ranging from 0.001 to 20 wt. % relative to the total weight of the ready-to-use composition and further for example, ranging from 0.005 to 10 wt. % relative to the total weight of the composition.

The medium that is suitable for dyeing may be, for instance, a suitable cosmetic medium comprising water or a mixture of water and at least one organic solvent, for example linear or branched C₁-C₄ lower alcohols, such as ethanol and isopropanol; the polyols and polyol ethers such as 2-butoxyethanol, propyleneglycol, monomethyl ether of propyleneglycol, monoethyl ether and monomethyl ether of diethyleneglycol, glycerol, as well as the aromatic alcohols such as benzyl alcohol or phenoxyethanol, and mixtures thereof.

In at least one embodiment, the at least one organic solvent is present in an amount ranging from 1 to 40 wt. % relative to the total weight of the dyeing composition, and further for example, ranging from 5 to 30 wt. % relative to the total weight of the dyeing composition.

According to at least one embodiment of the present disclosure, the dyeing composition further comprises at least one cosmetic additive chosen from antioxidants, penetrants, sequestering agents, perfumes, buffers, dispersants, surfactants, conditioners, film-forming agents, polymers, ceramides, preservatives, lustre agents, opacifiers, vitamins and provitamins.

The above additives are present in an amount, for each of them, ranging from 0.01 to 20 wt. % relative to the total weight of the composition.

The pH of the composition according to the present disclosure ranges from 3 to 12, for instance from 5 to 11. The pH can be adjusted to the desired value by means of acidifying or alkalizing agents usually employed in the dyeing of keratin fibers or alternatively by means of conventional buffering systems.

Among the acidifying agents, non-limiting mention may be made of, as examples, inorganic or organic acids other than the dicarboxylic acids such as hydrochloric acid, orthophosphoric acid, sulphuric acid, carboxylic acids such as acetic acid, tartaric acid, citric acid, lactic acid, and sulphonic acids.

Among the alkalizing agents non-limiting mention may be made of, as examples: ammonia, alkali metal carbonates, alkanolamines such as the mono-, di- and triethanolamines as well as their derivatives, the hydroxides of sodium or of potassium and the compounds of formula:

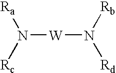

in which W is a propylene residue optionally substituted by at least one radical chosen from hydroxyl and C₁-C₄ alkyl radicals; Rₐ, R_{b}, R_{c} and R_{d}, which may be identical or different, are chosen from: hydrogen, C₁-C₄ alkyl radicals, and C₁-C₄ hydroxyalkyl radicals.

Of course, a person skilled in the art will ensure that the additive or additives, additional oxidation bases, oxidation couplers and direct dyes are selected in such a way that the beneficial properties intrinsically associated with the oxidation dyeing composition according to the present disclosure are not, or substantially are not, adversely affected by the additions that are envisaged.

The dyeing composition according to the present disclosure can be in various forms, such as in the form of liquids, creams, gels, or in any other form that is suitable for carrying out dyeing of keratin fibers, such as human hair.

The present disclosure further relates to a method of dyeing of keratin fibers in which the composition of the present disclosure, as defined above, is applied to the fibers, and the color is developed by means of an oxidizing agent. The color can be developed at acid, neutral or alkaline pH. According to at least one embodiment, the oxidizing agent can be added to the composition of the present disclosure at the exact moment of use. The composition as disclosed herein can be applied in the form of an oxidizing composition that contains it, or the oxidizing composition may be applied simultaneously or sequentially with the composition of the present disclosure.

As oxidizing agents, non-limiting mention may be made of: hydrogen peroxide, urea peroxide, bromates of alkali metals, per-salts such as perborates and persulphates, peracids and oxidase enzymes among which non-limitng mention may be made of: the peroxidases, 2-electron oxido-reductases such as the uricases and 4-electron oxygenases such as the laccases. In at least one embodiment, hydrogen peroxide is used.

According to at least one embodiment, the composition as disclosed herein is mixed, for instance at the moment of use, with a composition comprising, in a medium that is suitable for dyeing, at least one oxidizing agent, wherein the at least one oxidizing agent is present in an amount sufficient for dye development. The mixture obtained is then applied to the keratin fibers. After a holding time of 3 to 50 minutes, for example 5 to 30 minutes, the keratin fibers are rinsed, washed with shampoo, rinsed again and then dried.

The oxidizing composition may comprise various additives that are used conventionally in compositions for hair dyeing, including but not limited to those additives as previously defined.

The pH of the oxidizing composition comprising the at least one oxidizing agent is such that after mixing with the dyeing composition, the pH of the resulting composition applied to the keratin fibers varies for example from 3 to 12, and even further, for instance from 5 to 11. It can be adjusted to the desired value by means of acidifying or alkalizing agents usually employed in the dyeing of keratin fibers and as defined above.

The ready-to-use composition that is finally applied to the keratin fibers can be in various forms, such as in the form of liquids, creams, gels or in any other form that is suitable for carrying out dyeing of keratin fibers, notably human, such as human hair.

The present application also relates to the use of the cosmetic composition according to the present disclosure comprising, in a medium that is suitable for dyeing, at least one para-phenylenediamine of formula (I) for the dyeing of keratin fibers, for instance human keratin fibers, such as the hair.

The present disclosure also relates to a multi-compartment device or dyeing “kit” in which a first compartment comprises a dyeing composition comprising a para-phenylenediamine of formula (I) and a second compartment comprises an oxidizing composition. This kit can be equipped with a means of delivering the desired mixture onto the hair, such as the kits described in French Patent FR-2 586 913.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLES OF SYNTHESIS

### Example 1

### Synthesis of N-(4-aminophenyl)-N-[(3-{[(4-aminophenyl)amino]methyl}cyclohexyl)methyl]amine tetrahydrochloride (2)

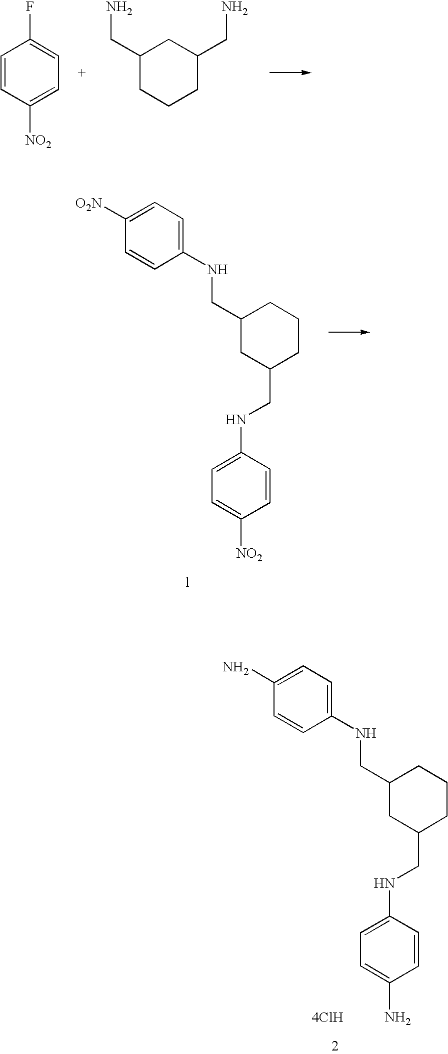

### Stage 1: synthesis of 4-nitro-N-[(3-{[(4-nitrophenyl)amino]methyl}cyclohexyl)methyl]aniline (1)

0.83 g of para-fluoronitrobenzene (2 eq.) was dissolved in 5 ml of DMSO. 1.2 equivalent of 1,3-bis(aminomethyl)cyclohexane and 4 equivalents of triethylamine were added to the solution. The reaction mixture was heated at 60° C. for 5 hours. The mixture was then poured onto crushed ice; a precipitate formed. The latter was filtered, washed with water, and then dried.

### Stage 2: synthesis of N-(4-aminophenyl)-N-[(3-{[(4-aminophenyl)amino]methyl}cyclohexyl)methyl]amine tetrahydrochloride (2)

The nitrogen-containing compound 1 was dissolved in 500 ml of ethanol in a hydrogenator. 10% of palladium on charcoal (50% water content) was added and the hydrogenator was charged with hydrogen. After reaction for 1 h 30 min, the palladium was filtered and 20 ml of ethanol-hydrochloric acid 3M then 300 ml of isopropyl ether were added to the filtrate. The precipitate obtained was filtered and recrystallized from ethanol-hydrochloric acid.

The proton and mass NMR spectra corresponded to the expected structure of the product.

### Example 2

### Synthesis of N-[3-(4-{3-[(4-aminophenyl)amino]propyl}piperazin-1-yl)propyl]benzene-1,4-diamine hexahydrochloride (4)

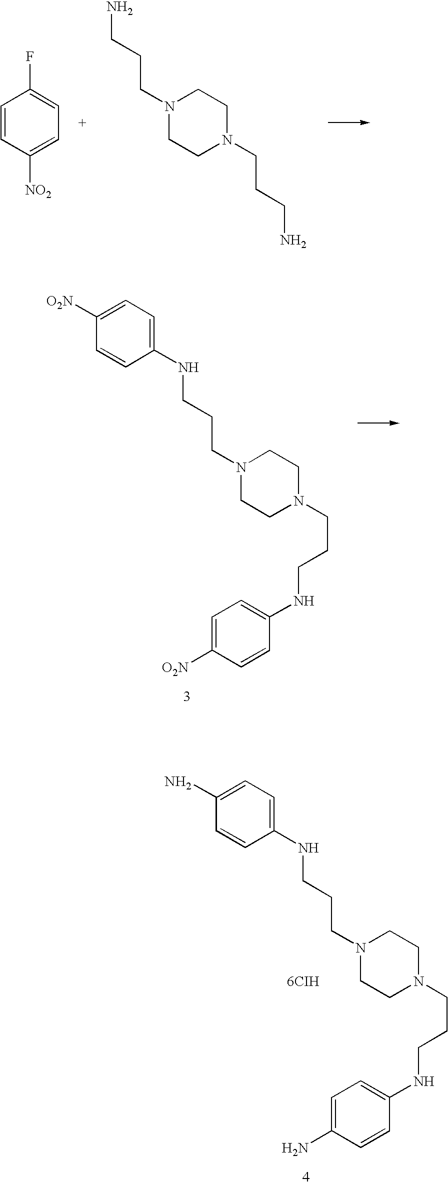

### Stage 1: synthesis of 4-nitro-N-[3-(4-{3-[(4-nitrophenyl)amino]propyl}piperazin-1-yl)propyl]aniline (3)

5 g of para-fluoronitrobenzene (2 eq.) was dissolved in 5 ml of DMSO. 1 equivalent of 1,4-bis(3-aminopropyl)-piperazine and 2.1 equivalents of triethylamine were added to the solution. The reaction mixture was heated at 60° C. for 24 hours. The mixture was then poured onto crushed ice; a precipitate formed. The latter was filtered, washed with water, and then dried.

### Stage 2: synthesis of N-[3-(4-{3-[(4-aminophenyl)amino]propyl}piperazin-1-yl)propyl]benzene-1,4-diamine hexahydrochloride (4)

The nitrogen-containing compound 3 was dissolved in 500 ml of ethanol in a hydrogenator. 10% of palladium on charcoal (50% water content) was added and the hydrogenator was charged with hydrogen. After reacting for 1 h 30 min, the palladium was filtered and 20 ml of ethanol-hydrochloric acid 3M then 300 ml of isopropyl ether were added to the filtrate. The precipitate obtained was filtered and recrystallized from ethanol-hydrochloric acid.

The proton and mass NMR spectra corresponded to the expected structure of the product.

### Example 3

### N-[(4-{[(4-aminophenyl)amino]methyl}cyclohexyl)methyl]benzene-1,4-diamine tetrahydrochloride (6)

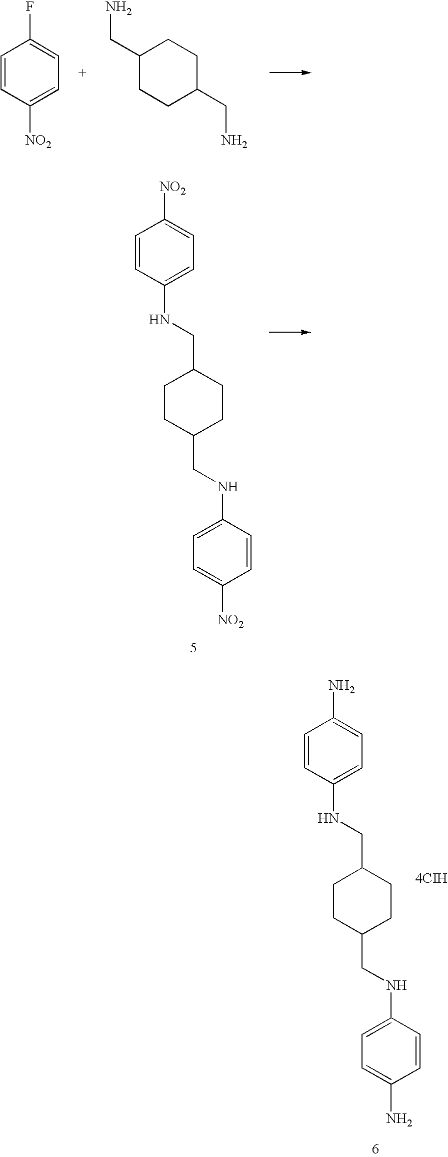

### Stage 1: synthesis of 4-nitro-N-[(4-{[(4-nitrophenyl)amino]methyl}cyclohexyl)methyl]aniline (5)

1.653 g of para-fluoronitrobenzene (2 eq.) was dissolved in 5 ml of DMSO. 1.2 equivalents of 1,4-bis(aminomethyl)cyclohexane and 4 equivalents of triethylamine were added to the solution. The reaction mixture was heated at 60° C. for 7 hours. The mixture was then poured onto crushed ice; a precipitate formed. The latter was filtered, washed with water, and then dried.

### Stage 2: synthesis of N-[(4-{[(4-aminophenyl)amino]methyl}cyclohexyl)methyl]benzene-1,4-diamine tetrahydrochloride (6)

1.2 g of 4-nitro-N-[(4-{[(4-nitrophenyl)amino]methyl}cyclohexyl)methyl]aniline was dissolved in 500 ml of ethanol in a hydrogenator. 0.5 g of palladium on charcoal (50% water content) was added and the hydrogenator was charged with hydrogen. After reacting for 1 h 30 min, the palladium was filtered and 15 equivalents of ethanol-hydrochloric acid 3M then 300 ml of isopropyl ether were added to the filtrate. The precipitate obtained was filtered.

The proton and mass NMR spectra corresponded to the expected structure of the product.

### Example 4

### Synthesis of N-(4-aminophenyl)-N-{3-[(4-aminophenyl)amino]cyclohexyl}amine tetrahydrochloride (8)

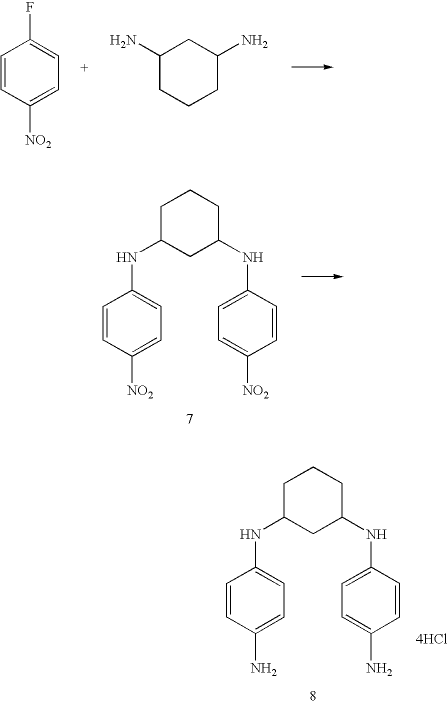

### Stage 1: synthesis of N-(4-nitrophenyl)-N-{3-[(4-nitrophenyl)amino]cyclohexyl}amine (7)

4 g of parafluoronitrobenzene (2 eq.) was dissolved in 13 ml of NMP. 1.2 equivalents of cyclohexane-1,3-diamine and 2 equivalents of trimethylamine were added to the solution. The reaction mixture was heated in a microwave oven (conditions: 200° C. for 15 minutes, 7 bar). The mixture was then poured into 50 ml of ethyl acetate, 75 ml of heptane is added; an oily deposit forms. The precipitated oil was recovered in ice water. It was filtered, washed with water, and then dried.

The proton and mass NMR spectra corresponded to the expected structure of the product.

### Stage 2: Synthesis of N-(4-aminophenyl)-N-{3-[(4-aminophenyl)amino]cyclohexyl}amine tetrahydrochloride (8)

Compound 7 obtained in the preceding stage was reduced with a boiling zinc/ammonium chloride/water/ethanol mixture. The resulting amine was isolated in the form of the tetrahydrochloride.

### EXAMPLES OF DYEING

### Examples 1 to 14

### Dyeing composition based on N-(4-aminophenyl)-N-[(3-{[(4-aminophenyl)amino]methyl}cyclohexyl)methyl]amine tetrahydrochloride (2)

### Examples 1 to 7

### Dyeing in an Acid Medium

The following dyeing compositions were prepared:

| | Example | | | | | | |
|---|---|---|---|---|---|---|---|
| | 1 | 2 | 3 | 4 | 5 | 6 | 7 |
| N-(4-aminophenyl)-N-[(3- | 10⁻³ | 10⁻³ | 10⁻³ | 10⁻³ | 10⁻³ | 10⁻³ | 10⁻³ |
| {[(4-aminophenyl)amino] | mol | mol | mol | mol | mol | mol | mol |
| methyl}cyclohexyl)methyl]amine | | | | | | | |
| tetrahydrochloride (2) | | | | | | | |
| Benzene-1,3-diol | 10⁻³ | | | | | | |
| | mol | | | | | | |
| 5-Amino-2-methyl-phenol | | 10⁻³ | | | | | |
| | | mol | | | | | |
| 1H-Indol-6-ol | | | 10⁻³ | | | | |
| | | | mol | | | | |
| 2-Amino-pyridin-3-ol | | | | 10⁻³ | | | |
| | | | | mol | | | |
| 3.6-Dimethyl-1H-pyrazolo[5,1-c][1,2,4]triazole | | | | | 10⁻³ | | |
| | | | | | mol | | |
| 2-(2,4-diamino-phenoxy)-ethanol | | | | | | 10⁻³ | |
| hydrochloride | | | | | | mol | |
| 3-Amino-2-chloro-6-methyl-phenol | | | | | | | 10⁻³ |
| hydrochloride | | | | | | | mol |
| Dyeing support (1) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (1) pH 7

| | | |
|---|---|---|
| 96° ethanol | 20.8 | g |
| Sodium metabisulphite, 35% aqueous solution | 0.23 | g M.A |
| Pentasodium salt of diethylene-triamine-pentaacetic | 0.48 | g M.A |
| acid, 40% aqueous solution | | |
| C₈-C₁₀ alkyl polyglycoside, 60% aqueous solution | 3.6 | g M.A |
| Benzyl alcohol | 2.0 | g |
| Polyethylene glycol with 8 ethylene oxide units | 3.0 | g |
| Na₂HPO₄ | 0.28 | g |
| KH₂PO₄ | 0.46 | g |

At the moment of use, each composition was mixed with an equal weight of hydrogen peroxide at 20 volumes (6 wt. %). A final pH of 7 was obtained.

Each mixture obtained was applied to locks of grey hair at 90% white. After a holding time of 30 minutes, the locks were rinsed, washed with a standard shampoo, rinsed again and then dried.

The shades obtained are shown in the following table:

| | Example | | | | | | |
|---|---|---|---|---|---|---|---|
| | 1 | 2 | 3 | 4 | 5 | 6 | 7 |
| Shade | deep | deep | deep | deep | deep | deep | deep |
| observed | grey | grey | brown | grey | grey | violet- | grey |
| | | | | | | blue | violet |

### Examples 8 to 14

### Dyeing in a Basic Medium

The following dyeing compositions were prepared:

| | Example | | | | | | |
|---|---|---|---|---|---|---|---|
| | 8 | 9 | 10 | 11 | 12 | 13 | 14 |
| N-(4-aminophenyl)-N-[(3- | 10⁻³ | 10⁻³ | 10⁻³ | 10⁻³ | 10⁻³ | 10⁻³ | 10⁻³ |
| {[(4-aminophenyl)amino] | mol | mol | mol | mol | mol | mol | mol |
| methyl}cyclohexyl)methyl]amine | | | | | | | |
| tetrahydrochloride (2) | | | | | | | |
| Benzene-1,3-diol | 10⁻³ | | | | | | |
| | mol | | | | | | |
| 5-Amino-2-methyl-phenol | | 10⁻³ | | | | | |
| | | mol | | | | | |
| 1H-Indol-6-ol | | | 10⁻³ | | | | |
| | | | mol | | | | |
| 2-Amino-pyridin-3-ol | | | | 10⁻³ | | | |
| | | | | mol | | | |
| 3.6-Dimethyl-1H-pyrazolo[5,1-c][1,2,4]triazole | | | | | 10⁻³ | | |
| | | | | | mol | | |
| 2-(2,4-Diamino-phenoxy)-ethanol | | | | | | 10⁻³ | |
| hydrochloride | | | | | | mol | |
| 3-Amino-2-chloro-6-methyl-phenol | | | | | | | 10⁻³ |
| hydrochloride | | | | | | | mol |
| Dyeing support (2) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (2) pH 9.5

| | |
|---|---|
| 96° ethanol | 20.8 g |
| Sodium metabisulphite, 35% aqueous solution | 0.23 g M.A |
| Pentasodium salt of diethylene-triamine-pentaacetic acid, | 0.48 g MA |
| 40% aqueous solution | |
| C₈-C₁₀ alkyl polyglycoside, 60% aqueous solution | 3.6 g M.A |
| Benzyl alcohol | 2.0 g |
| Polyethylene glycol with 8 ethylene oxide units | 3.0 g |
| NH₄Cl | 4.32 g |
| Ammonia at 20% NH₃ | 2.94 g |

At the moment of use, each composition was mixed with an equal weight of hydrogen peroxide at 20 volumes (6 wt. %). A final pH of 9.5 was obtained.

Each mixture obtained was applied to locks of grey hair at 90% white. After a holding time of 30 minutes, the locks were rinsed, washed with a standard shampoo, rinsed again and then dried.

The shades obtained are shown in the following table:

| | Example | | | | | | |
|---|---|---|---|---|---|---|---|
| | 8 | 9 | 10 | 11 | 12 | 13 | 14 |
| Shade | deep | deep | deep | deep | deep | deep | deep |
| observed | red- | violet | red- | grey | red | grey | violet- |
| | brown | | brown | red | | violet- | blue |
| | | | | | | blue | |

### Examples 15 to 28

### Dyeing composition based on N-[3-(4-{3-[(4-aminophenyl)amino]propyl}piperazin-1-yl)propyl]benzene-1,4-diamine hexahydrochloride (4)

### Examples 15 to 21

### Dyeing in an Acid Medium

The following dyeing compositions were prepared:

| | Example | | | | | | |
|---|---|---|---|---|---|---|---|
| | 15 | 16 | 17 | 18 | 19 | 20 | 21 |
| N-[3-(4-{3-[(4-aminophenyl)amino] | 10⁻³ | 10⁻³ | 10⁻³ | 10⁻³ | 10⁻³ | 10⁻³ | 10⁻³ |
| propyl}piperizin-1-yl)propyl]benzene- | mol | mol | mol | mol | mol | mol | mol |
| 1,4-diamine hexahydrochloride (4) | | | | | | | |
| Benzene-1,3-diol | 10⁻³ | | | | | | |
| | mol | | | | | | |
| 5-Amino-2-methyl-phenol | | 10⁻³ | | | | | |
| | | mol | | | | | |
| 1H-Indol-6-ol | | | 10⁻³ | | | | |
| | | | mol | | | | |
| 2-Amino-pyridin-3-ol | | | | 10⁻³ | | | |
| | | | | mol | | | |
| 3.6-Dimethyl-1H-pyrazolo[5,1-c][1,2,4]triazole | | | | | 10⁻³ | | |
| | | | | | mol | | |
| 2-(2,4-Diamino-phenoxy)-ethanol | | | | | | 10⁻³ | |
| hydrochloride | | | | | | mol | |
| 3-Amino-2-chloro-6-methyl-phenol | | | | | | | 10⁻³ |
| hydrochloride | | | | | | | mol |
| Dyeing support (1) | (*) | (*) | (*) | (*) | (*) | (*) | (*) |
| Demineralized water q.s.f. | 100 g | 100 g | 100 g | 100 g | 100 g | 100 g | 100 g |

At the moment of use, each composition was mixed with an equal weight of hydrogen peroxide at 20 volumes (6 wt. %). A final pH of 7 was obtained.

Each mixture obtained was applied to locks of grey hair at 90% white. After a holding time of 30 minutes, the locks were rinsed, washed with a standard shampoo, rinsed again and then dried.

The shades obtained are shown in the following table:

| | Example | | | | | | |
|---|---|---|---|---|---|---|---|
| | 15 | 16 | 17 | 18 | 19 | 20 | 21 |
| Shade | deep | deep | deep | deep | deep | deep | deep |
| observed | grey | grey | grey | brown | grey | blue | violet |
| | | violet | red | red | violet- | | |
| | | | | | red | | |

### Examples 22 to 28

### Dyeing in a Basic Medium

The following dyeing compositions were prepared:

| | Example | | | | | | |
|---|---|---|---|---|---|---|---|
| | 22 | 23 | 24 | 25 | 26 | 27 | 28 |
| N-[3-(4-{3-[(4-aminophenyl)amino] | 10⁻³ | 10⁻³ | 10⁻³ | 10⁻³ | 10⁻³ | 10⁻³ | 10⁻³ |
| propyl}piperizin-1-yl)propyl]benzene- | mol | mol | mol | mol | mol | mol | mol |
| 1,4-diamine hexahydrochloride (4) | | | | | | | |
| Benzene-1,3-diol | 10⁻³ | | | | | | |
| | mol | | | | | | |
| 5-Amino-2-methyl-phenol | | 10⁻³ | | | | | |
| | | mol | | | | | |
| 1H-Indol-6-ol | | | 10⁻³ | | | | |
| | | | mol | | | | |
| 2-Amino-pyridin-3-ol | | | | 10⁻³ | | | |
| | | | | mol | | | |
| 3,6-Dimethyl-1H-pyrazolo[5,1-c][1,2,4]triazole | | | | | 10⁻³ | | |
| | | | | | mol | | |
| 2-(2,4-Diamino-phenoxy)-ethanol | | | | | | 10⁻³ | |
| hydrochloride | | | | | | mol | |
| 3-Amino-2-chloro-6-methyl-phenol | | | | | | | 10⁻³ |
| hydrochloride | | | | | | | mol |
| Dyeing support (2) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (2) as defined previously

At the moment of use, each composition was mixed with an equal weight of hydrogen peroxide at 20 volumes (6 wt. %). A final pH of 9.5 was obtained.

Each mixture obtained was applied to locks of grey hair at 90% white. After a holding time of 30 minutes, the locks were rinsed, washed with a standard shampoo, rinsed again and then dried.

The shades obtained are shown in the following table:

| | Example | | | | | | |
|---|---|---|---|---|---|---|---|
| | 22 | 23 | 24 | 25 | 26 | 27 | 28 |
| Shade | deep | deep | deep | deep | deep | deep | deep |
| observed | brown | violet | red | brown | shade | violet- | violet |
| | orange | | | red | | blue | |

What is claimed is:

1. A compound chosen from para-phenylenediamines of formula (I) and the addition salts thereof: 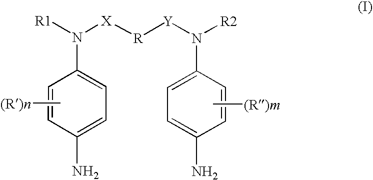
wherein:
R is a saturated ring with 4 to 7 ring members, optionally comprising at least one atom chosen from nitrogen and oxygen atoms, and optionally substituted,
R₁ and R₂ are, independently of one another, chosen from:
hydrogen,
linear or branched C₁-C₆ alkyl radicals,
linear or branched C₁-C₆ alkyl radicals substituted by at least one radical chosen from hydroxy, C₁-C₄ alkoxy, amino, C₁-C₄ monoalkylamino, and C₁-C₄ dialkylamino radicals,
X and Y are, independently of one another, chosen from linear or branched C₁-C₁₀ alkylene radicals and a covalent bond,
R′ and R″ are, independently of one another, chosen from C₁-C₆ alkyl radicals, C₁-C₆ alkoxy radicals, C₁-C₆ hydroxy-alkoxy radicals, C₁-C₆ alkoxy(C₁-C₆)alkyl radicals, C₁-C₆ mono- or poly-hydroxy alkyl radicals,
n and m are, independent of one another, integers ranging from 0 to 4;
with the exception of the following compound:

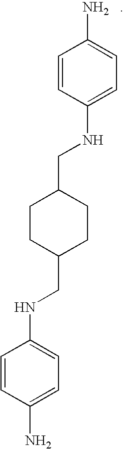

2. The para-phenylenediamine compound according to claim 1, wherein R is a saturated cyclic radical.

3. The para-phenylenediamine compound according to claim 2, wherein R further comprises a nitrogen atom.

4. The para-phenylenediamine compound according to claim 1, wherein R is a cyclic radical substituted by a C₁-C₄ alkyl radical.

5. The para-phenylenediamine compound according to claim 1, wherein R₁ and R₂ are independently chosen from hydrogen and optionally substituted C₁-C₄ alkyl groups.

6. The para-phenylenediamine compound according to claim 1, wherein X and Y are independently chosen from C₁-C₃ alkylene radicals and a covalent bond.

7. The para-phenylenediamine compound according claim 1, wherein n and m are each an integer, independently chosen from 0 and 1.

8. The para-phenylenediamine compound according to claim 1, wherein the acid-addition salts of the para-phenylenediamines of formula (I) are chosen from salts of hydrochloric acid, hydrobromic acid, sulphuric acid, citric acid, succinic acid, tartaric acid, lactic acid, para-toluene-sulphonic acid, benzene-sulphonic acid, phosphoric acid and acetic acid, these compounds optionally being in the form of solvates.

9. The para-phenylenediamine compound according to claim 1, chosen from:

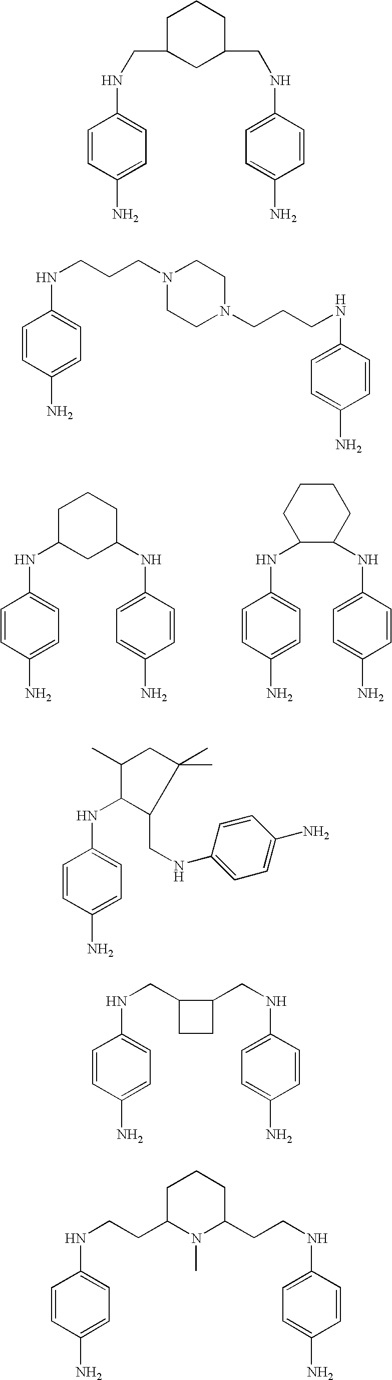

10. A dyeing composition comprising, in a medium that is suitable for dyeing, at least one oxidation base chosen from para-phenylenediamine compounds of formula (I) and addition salts thereof 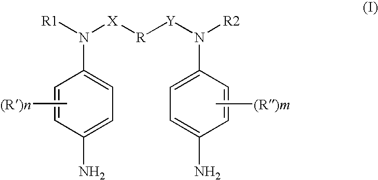
wherein:
R is a saturated ring with 4 to 7 ring members, optionally comprising at least one atom chosen from nitrogen and oxygen atoms, and optionally substituted,
R₁ and R₂ are independently chosen from:
hydrogen,
linear or branched C₁-C₆ alkyl radicals,
linear or branched C₁-C₆ alkyl radicals substituted by at least one radical chosen from hydroxy, C₁-C₄ alkoxy, amino, C₁-C₄ monoalkylamino, and C₁-C₄ dialkylamino radicals,
X and Y are independently chosen from linear or branched C₁-C₁₀ alkylene radicals and a covalent bond,
R′ and R″ are independently chosen from C₁-C₆ alkyl radicals, C₁-C₆ alkoxy radicals, C₁-C₆ hydroxy-alkoxy radicals, C₁-C₆ alkoxy(C₁-C₆)alkyl radicals, C₁-C₆ mono- or poly-hydroxy alkyl radicals,
n and m are each an integer, independently chosen from 0 to 4.

11. The composition according to claim 10, wherein the at least one oxidation base is present in an ranging from 0.0001% to 20 wt. % relative to the total weight of the composition.

12. The composition according to claim 11, wherein the at least one oxidationn base is present in an amount ranging from 0.01% to 10% wt. % relative to the total weight of the composition.

13. The composition according to claim 10, further comprising at least one oxidation coupler.

14. The composition according to claim 13, wherein the at least one oxidation coupler is chosen from meta-phenylenediamines, meta-aminophenols, meta-diphenols, naphthalenic couplers, heterocyclic couplers, and addition salts thereof.

15. The composition according to claim 10, further comprising at least one additional oxidation base different from the oxidation bases of formula (I).

16. The composition according to claim 15, wherein the at least one additional oxidation base is chosen from para-phenylenediamines, bis-phenylalkylenediamines, para-aminophenols, ortho-aminophenols, heterocyclic bases and addition salts thereof.

17. The composition according to claim 10, further comprising at least one direct dye.

18. The composition according to claim 10, wherein the medium suitable for dyeing comprises water.

19. The composition according to claim 18, wherein the medium suitable for dyeing further comprises at least one organic solvent.

20. The composition according to claim 19, wherein the at least one organic solvent is chosen from linear or branched C₁-C₄ lower alcohols, from aromatic alcohols and from mixtures thereof.

21. The composition according to claim 10, further comprising at least one cosmetic additive chosen from antioxidants, penetrants, sequestering agents, perfumes, buffers, dispersants, surfactants, conditioners, film-forming agents, polymers, ceramides, preservatives, lustre agents, opacifiers, and vitamins or provitamins.

22. The composition according to claim 21, wherein the at least one cosmetic additive is, for each additive, present in an amount ranging from 0.01 to 20 wt. % relative to the total weight of the composition.

23. The composition according to claim 10, further comprising at least one oxidizing agent.

24. A method for dyeing keratin fibers, comprising:
applying at least one dyeing composition to the keratin fibers in the presence of an oxidizing agent for a sufficient time for development of a desired coloration,
wherein the at least one dyeing composition comprises, in a medium that is suitable for dyeing, at least one oxidation base chosen from para-phenylenediamine compounds of formula (I) and addition salts thereof 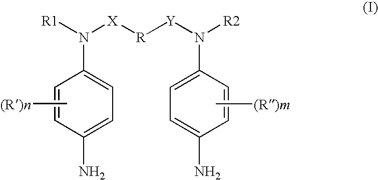
wherein:
R is a saturated ring with 4 to 7 ring members, optionally comprising at least one atom chosen from nitrogen and oxygen atoms, and optionally substituted,
R₁ and R₂ are independently chosen from:
hydrogen,
linear or branched C₁-C₆ alkyl radicals,
linear or branched C₁-C₆ alkyl radicals substituted by at least oneradical chosen from hydroxy, C₁-C₄ alkoxy, amino, C₁-C₄ monoalkylamino, and C₁-C₄ dialkylamino radicals,
X and Y are independently chosen from linear or branched C₁-C₁₀ alkylene radicals and a covalent bond,
R′ and R″ are independently chosen from C₁-C₆ alkyl radicals, C₁-C₆ alkoxy radicals, C₁-C₆ hydroxy-alkoxy radicals, C₁-C₆ alkoxy(C₁-C₆)alkyl radicals, C₁-C₆ mono- or poly-hydroxy alkyl radicals,
n and m are each an integer, independently chosen from 0 to 4.

25. A multi-compartment kit, comprising:
a first compartment comprising a dyeing composition for the dyeing of keratin fibers, wherein the dyeing composition comprises, in a medium that is suitable for dyeing, at least one oxidation base chosen from para-phenylenediamine compounds of formula (I) and addition salts thereof 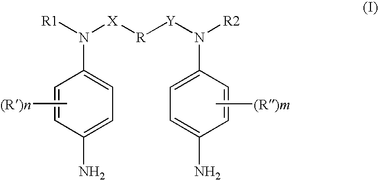
wherein:
R is a saturated ring with 4 to 7 ring members, optionally comprising at least one atom chosen from nitrogen and oxygen atoms, and optionally substituted,
R₁ and R₂ are independently chosen from:
hydrogen,
linear or branched C₁-C₆ alkyl radicals,
linear or branched C₁-C₆ alkyl radicals substituted by at least oneradical chosen from hydroxy, C₁-C₄ alkoxy, amino, C₁-C₄ monoalkylamino, and C₁-C₄ dialkylamino radicals,
X and Y are independently chosen from linear or branched C₁-C₁₀ alkylene radicals and a covalent bond,
R′ and R″ are independently chosen from C₁-C₆ alkyl radicals, C₁-C₆ alkoxy radicals, C₁-C₆ hydroxy-alkoxy radicals, C₁-C₆ alkoxy(C₁-C₆)alkyl radicals, C₁-C₆ mono- or poly-hydroxy alkyl radicals,
n and m are each an integer, independently chosen from 0 to 4; and
a second compartment comprising at least one oxidizing agent.

26. A nitro compound of formula (II): 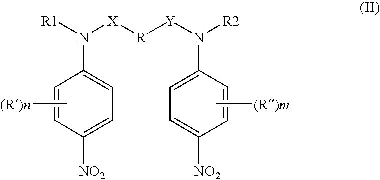
wherein:
R is a saturated ring with 4 to 7 ring members, optionally comprising at least one atom chosen from nitrogen and oxygen atoms, and optionally substituted,
R₁ and R₂ are independently chosen from:
hydrogen,
linear or branched C₁-C₆ alkyl radicals,
linear or branched C₁-C₆ alkyl radicals substituted by at least oneradical chosen from hydroxy, C₁-C₄ alkoxy, amino, C₁-C₄ monoalkylamino, and C₁-C₄ dialkylamino radicals,
X and Y are independently chosen from linear or branched C₁-C₁₀ alkylene radicals and a covalent bond,
R′ and R″ are independently chosen from C₁-C₆ alkyl radicals, C₁-C₆ alkoxy radicals, C₁-C₆ hydroxy-alkoxy radicals, C₁-C₆ alkoxy(C₁-C₆)alkyl radicals, C₁-C₆ mono- or poly-hydroxy alkyl radicals,
n and m are each an integer, independently chosen from 0 to 4; and
with the exception of the following compounds:

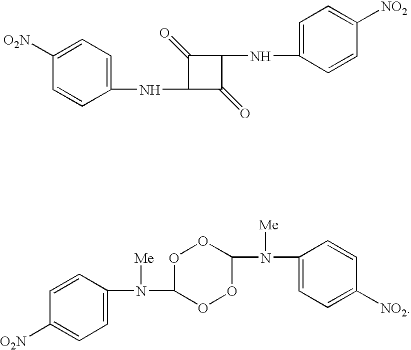

27. A method of synthesizing a para-phenylenediamine compound of formula (I): 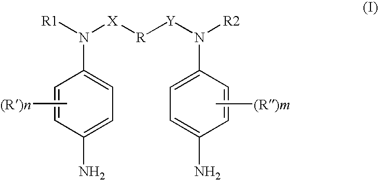
wherein:
R is a saturated ring with 4 to 7 ring members, optionally comprising at least one atom chosen from nitrogen and oxygen atoms, and optionally substituted,
R₁ and R₂ are independently chosen from:
hydrogen,
linear or branched C₁-C₆ alkyl radicals,
linear or branched C₁-C₆ alkyl radicals substituted by at least oneradical chosen from hydroxy, C₁-C₄ alkoxy, amino, C₁-C₄ monoalkylamino, and C₁-C₄ dialkylamino radicals,
X and Y are independently chosen from linear or branched C₁-C₁₀ alkylene radicals and a covalent bond,
R′ and R″ are independently chosen from C₁-C₆ alkyl radicals, C₁-C₆ alkoxy radicals, C₁-C₆ hydroxy-alkoxy radicals, C₁-C₆ alkoxy(C₁-C₆)alkyl radicals, C₁-C₆ mono- or poly-hydroxy alkyl radicals, and
n and m are each an integer, independently chosen from 0 to 4;
said method comprising reducing the nitrogen-containing compounds of formula (II): 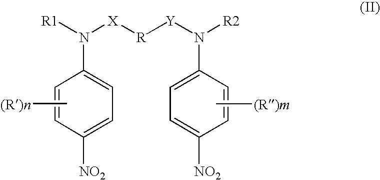
wherein:
R is a saturated ring with 4 to 7 ring members, optionally comprising at least one atom chosen from nitrogen and oxygen atoms, and optionally substituted,
R₁ and R₂ are independently chosen from:
hydrogen,
linear or branched C₁-C₆ alkyl radicals,
linear or branched C₁-C₆ alkyl radicals substituted by at least oneradical chosen from hydroxy, C₁-C₄ alkoxy, amino, C₁-C₄ monoalkylamino, and C₁-C₄ dialkylamino radicals,
X and Y are independently chosen from linear or branched C₁-C₁₀ alkylene radicals and a covalent bond,
R′ and R″ are independently chosen from C₁-C₆ alkyl radicals, C₁-C₆ alkoxy radicals, C₁-C₆ hydroxy-alkoxy radicals, C₁-C₆ alkoxy(C₁-C₆)alkyl radicals, C₁-C₆ mono- or poly-hydroxy alkyl radicals,
n and m are each an integer, independently chosen from 0 to 4;
with the exception of the following compound: [chemical structure]
